(19) United States
(12) Patent Application Publication
(10) Pub. No.: US 2022/0202409 A1
(43) Pub. Date: Jun. 30, 2022

(54) EYELID MANIPULATION DEVICE AND METHODS OF USE

(71) Applicants: Kerry Salsberg, Toronto (CA); Jordan Friedman, Toronto (CA)

(72) Inventors: Kerry Salsberg, Toronto (CA); Jordan Friedman, Toronto (CA)

(21) Appl. No.: 17/551,408

(22) Filed: Dec. 15, 2021

Related U.S. Application Data
(60) Provisional application No. 63/130,542, filed on Dec. 24, 2020.

(51) Int. Cl. A61B 17/02; A61F 9/007

(52) U.S. Cl. CPC A61B 17/0231; A61F 9/0079; A61F 9/00772

(57) ABSTRACT

An eyelid manipulation device includes a rigid contact portion dimensioned to be insinuated between an eyelid and a globe, the contact portion having an upwardly-facing surface opposite a downwardly-facing surface, each of the surfaces extending between a distal side and a proximal side of the contact portion; and a rigid stem integral with and extending upwardly from the proximal side of the contact portion. In embodiments, the stem is connectable to a handle. In other embodiments, the stem is elongated, thereby serving as a handle. The eyelid manipulation device may be used during treatment of Meibomian glands or otherwise during treatment of dry eye symptoms.

### CROSS-REFERENCE TO RELATED APPLICATION

This application claims priority to U.S. Provisional Patent Application Ser. No. 63/130,542 filed on Dec. 24, 2020, the contents of which are incorporated herein by reference.

### FIELD OF THE INVENTION

The following relates generally to medical devices and techniques and more particularly to an eyelid manipulation device for use during treatment of eyelids and eyes.

### BACKGROUND OF THE INVENTION

Radio Frequency (RF) is an aesthetic technique that uses radio frequency energy to heat skin to regenerate collagen and elastin to reduce the appearance of forehead wrinkles, frown lines, crow's feet, and smile lines. An off-label procedure is to use RF to reduce dry eye symptoms by heating the Meibomian glands located in the upper and lower lids. Meibomian glands are oil-producing glands that help protect the tears from evaporation. When they become clogged or if they are not functioning properly, the eye suffers from a degenerative disease called Meibomian Gland Dysfunction (MGD), which is often associated with dry eye syndrome.

Commonly, RF application to reduce dry-eye symptoms involves using an RF hand piece to apply penetrative heat and vibration to the Meibomian glands, providing mechanical expression and stimulation of the glands. Protocol for RF treatment for MGD requires a corneal shield placed under the upper and lower lids to protect the cornea from heat and potential contact injury from the downward pressure of the RF hand piece.

United States Patent Application Publication No. 2017/0071790 to Grenon et al. discloses methods and apparatuses for treatment of MGD.

United States Patent Application Publication No. 2020/0069468 to Litherland et al. discloses devices and methods for treating eyelids and expressing occlusions in the Meibomian glands of the eyelids.

Current corneal shields proposed for dry eye treatment tend to have a number of shortcomings. For example, they tend to be produced in one-size-fits-all format, and to be expensive. Furthermore, current corneal shields, due to their bulk and their large size relative to the treatment region and space between eyelids, can be difficult to insert between the eyelid and the globe. Once finally in place, current corneal shields can be difficult to manipulate, and can accordingly be uncomfortable for a patient because when manipulation is attempted they can easily inadvertently pinch skin of the eyelid. Furthermore, due to their bulk with respect to the size of the eye, it can be difficult to work on specific regions of an eyelid—for example to manually target counter pressure to facilitate expression of the Meibomian glands in a particular treatment region. In addition, due to their limited mobility once positioned between the eyelid and the eyeball, it can be difficult to manipulate current corneal shields to treat lid margins.

It is an object of the following description to address the disadvantages referred to above.

### SUMMARY OF THE INVENTION

According to an aspect of this description, there is provided an eyelid manipulation device comprising: a rigid contact portion dimensioned to be insinuated between an eyelid and a globe, the contact portion having an upwardly-facing surface opposite a downwardly-facing surface, each of the surfaces extending between a distal side and a proximal side of the contact portion; and a rigid stem integral with and extending upwardly from the proximal side of the contact portion.

In an embodiment, at least the exterior of the eyelid manipulation device is made of at least one non-conductive material.

In an embodiment, the at least one non-conductive material comprises a medical grade material.

In an embodiment, the at least one medical grade material is selected from the group consisting of silicone and polyethylene.

In an embodiment, the stem is connectable to a handle.

According to another aspect, there is provided an eyelid manipulation system comprising: the eyelid manipulation device; and an elongate handle removably connectable to the rigid stem.

In an embodiment, at least the exterior of the handle is formed of at least one non-conducting material.

According to another aspect, there is provided a use of the eyelid manipulation system for treatment of Meibomian glands.

According to another aspect, there is provided a use of the eyelid manipulation system for treatment of dry eye symptoms.

In an embodiment of the eyelid manipulation device, the stem is elongated, thereby serving as a handle. The elongated stem eyelid manipulation device may itself be used for treatment of Meibomian glands, and/or for treatment of dry eye symptoms.

Other aspects and advantages will be apparent upon reading the following.

### BRIEF DESCRIPTION OF THE DRAWINGS

Embodiments of the invention will now be described with reference to the appended drawings in which:

FIG. 1 is a rear perspective rendered view of an eyelid manipulation device (EMD) above the globe of an eye and connected to a handle, according to an embodiment;

FIG. 2 is a front perspective rendered view of the EMD and handle of FIG. 1;

FIG. 3 is another front perspective rendered view of the EMD and handle of FIG. 1, with the EMD shown separated from the handle, according to an embodiment;

FIG. 4 is a front perspective wireframe view of the EMD of FIG. 1, with the EMD shown connected to the handle;

FIG. 5 is a front perspective wireframe view of the EMD of FIG. 1, with the EMD shown separated from the handle;

FIG. 6A is a top plan wireframe view of the EMD of FIG. 1, with the EMD connected to the handle;

FIG. 6B is a side view of the EMD of FIG. 6A, shown sectioned along the line A-A in FIG. 6A;

FIG. 7A is a top plan wireframe view of the EMD of FIG. 1, with the EMD shown separated from the handle;

FIG. 7B is a side view of the EMD of FIG. 7A, shown sectioned along the line B-B in FIG. 7A;

FIG. 8A is a front perspective rendered view of the EMD of FIG. 1, in isolation;

FIG. 8B is a front perspective wireframe view of EMD of FIG. 1, in isolation;

FIG. 9A is a side elevation rendered view of EMD of FIG. 1, in isolation;

FIG. 9B is a side elevation wireframe view of the EMD of FIG. 1, in isolation;

FIG. 9C is a side section rendered view of the EMD of FIG. 1, in isolation;

FIG. 9D is a side section wireframe view of the EMD of FIG. 1, in isolation;

FIG. 10A is a top rendered view of the EMD of FIG. 1, in isolation;

FIG. 10B is a top wireframe view of the EMD of FIG. 1, in isolation;

FIG. 11A is a bottom rendered view of the EMD of FIG. 1, in isolation;

FIG. 11B is a bottom wireframe view of the EMD of FIG. 1, in isolation;

FIG. 12A is a distal end rendered view of the EMD of FIG. 1, in isolation;

FIG. 12B is a distal end wireframe view of the EMD of FIG. 1, in isolation;

FIGS. 13A, 13B and 13C are side sectional views of the eyelid and globe of a leftward-facing individual, with the EMD (handle and user not shown) being insinuated between the eyelid and the globe and then being manipulated to progressively lift a region of the eyelid upwardly and away from the globe for a treatment of that region; and

FIG. 14 is a front perspective rendered view of an EMD, according to another embodiment.

### DETAILED DESCRIPTION

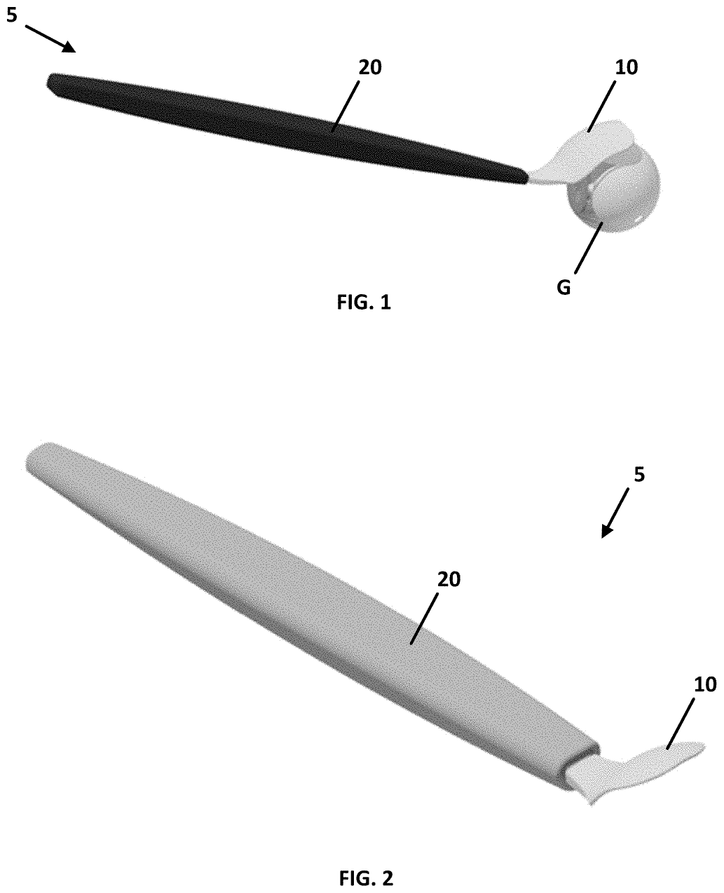

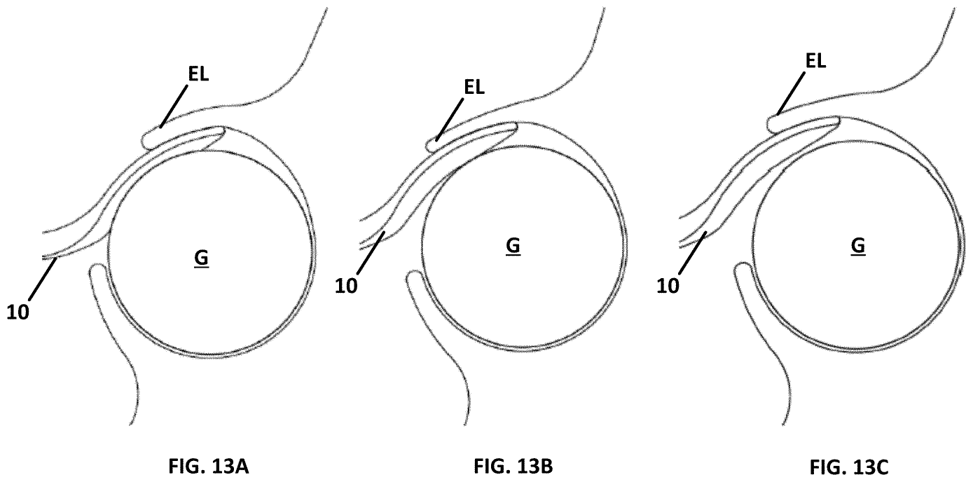

FIG. 1 is a rear perspective rendered view of an eyelid manipulation system 5 including an eyelid manipulation device (EMD) 10 above the globe G of an eye and connected to a handle 20, according to an embodiment. FIG. 2 is a front perspective rendered view of EMD 10 and handle 20. In this embodiment, EMD 10 has a rigid contact portion 12 dimensioned to be insinuated between an eyelid EL and the globe G (as shown in FIGS. 13A, 13B and 13C). Contact portion 12 includes an upwardly facing surface 13 opposite a downwardly facing surface 14, each of the surfaces extending between a distal side 15 and a proximal side 16 of the contact portion 12. EMD 10 also has rigid stem 17 that is integral with and extends upwardly from proximal side 16 of contact portion 12. In this embodiment, rigid stem 17 can be inserted into and removed from handle 20, so that EMD 10 itself can be disposed of or, if desired, sanitized without necessarily disposing of or sanitizing handle 20. Handle 20 may, if appropriate, be re-used. Contact portion 12 is referred to as such, as it is the portion of EMD 10 that generally comes into contact with globe G as it is inserted between globe G and eyelid EL, and that contacts regions of eyelid EL during manipulation. Stem 17 extends upwardly to extend away from globe G during use. In this embodiment, handle 20 to which stem 17 is connected extends linearly from stem 17.

In this embodiment, at least the exterior of the eyelid manipulation device is made of one or more non-conductive materials, such as medical grade silicone, medical grade polymer such as polyethylene, and/or some other material. Other non-conductive materials may be used provided that they are appropriate in an eyelid manipulation device such as is disclosed herein. The non-conductive material resists transfer of heat emitted from a radiofrequency treatment device or another heat treatment device that is incident on eyelid manipulation device 10 through to the eyelid EL, globe G, a cheek or other feature of the person whose eyelids are being treated via eyelid manipulation device 10. In embodiments, a core or backbone of the eyelid manipulation device, or components thereof, might be made of a conductive material that is itself coated with a non-conductive material, thereby to provide an exterior that resists the heat transfer to the person being treated. It will be appreciated that although a coated conductive core eyelid manipulation device could be useful for providing rigidity while sufficiently resisting such heat transfer, such a device could be more complex to manufacture than an eyelid manipulation device made or molded from a single material.

Furthermore, in this embodiment at least the exterior of handle 20 is formed of a non-conductive material, such that an eyelid manipulation system 5 comprising eyelid manipulation device 10 and handle 20, as a whole, can resist transfer of any heat to the person being treated.

In this description, rigidity refers to contact portion 12 and stem 17 being generally inflexible with respect to each other during manipulation, so that manipulative force imparted via handle 10 to stem 17 translates predictably for a user to corresponding force applied via contact portion 12 to the region of the eyelid EL to be manipulated. It is to be understood that there may be some flexibility or yield to material that may coat the surfaces of at least the contact portion of the EMD—such as medical grade silicone. Furthermore, EMD 10 may be flexible or bendable upon application of an amount of force that significantly exceeds that generally imparted to eyelid manipulation device 10 when it is insinuated between an eyelid EL and a globe G and is being used to manipulate eyelid EL. Therefore, while eyelid manipulation device 10 might be deliberately flexed or bent if desired and if its material permits, it is to be understood that it is useful for handle 20, stem 17 and contact portion 12 to behave, during and for the purpose of manipulation, as a single generally-inflexible unit so that the user's delicate manipulations of handle 20 are translated predictably into just as delicate manipulations of contact portion 12.

In this embodiment, EMD 10 is sized and shaped to be insinuated between an eyelid EL and globe G, so that the eyelid EL can be manipulated in various ways during a treatment while the underlying globe G is being shielded by EMD 10 from any incident radiation. Such manipulation may include lifting eyelid EL at selected locations along eyelid EL thereby to lift eyelid EL out of a rest position (which is against globe G) to which eyelid EL is normally biased. Lifting eyelid EL against its bias imparts pressure on the underside of eyelid EL, which pressure—particularly when targeted using EMD 10—can be useful for assisting with manipulating Meibomian glands as explained above.

Contact portion 12 of EMD 10 is generally narrower between its left and right sides with respect to globe G than are corneal shields disclosed in the prior art. This enables a user to focus on a particular region of eyelid EL for manipulation at any given time. Furthermore, contact portion 12 and stem 17 are arranged with respect to each other to permit EMD 10 to be used to manipulate individual regions of just one eyelid EL (of two associated with a given eye) at a time. In particular, stem 17 of EMD 10—and accordingly handle 20 with which it is connected during use—extends from the proximal end of contact region 12, rather than from a top surface as in known corneal shields. For example, known corneal shields having a handle extending from the top surface of a corneal shield leave a portion of the corneal shield behind the handle. This portion of the corneal shield behind the handle can interfere with an edge of an eyelid while the user's focus is on the other eyelid, potentially pinching the eyelid. In contrast, EMD 10 and handle 20 of FIG. 1 enables the “heel” of the overall combination to be aligned with the main axis of handle 20, affording a user greater control and focus for manipulation of the eyelid EL being worked on, and less inadvertent pinching of an eyelid associated with the same eye but that is not—at that moment—being worked on. Furthermore, EMD 10 is easier than prior art devices to insert between a single eyelid EL and globe G. This is because, due to their configuration, prior art devices must generally be inserted beneath both eyelids even while only one eyelid is being treated.

Contact portion 12 of EMD 10 is of sufficient thickness to serve as a corneal RF shield, thereby to sufficiently shield a globe G from incident RF radiation and/or heat that may be applied to eyelid EL from above. In this embodiment, stem 17 is also sufficient to provide some shielding should the RF radiation and/or heat be incident on stem 17.

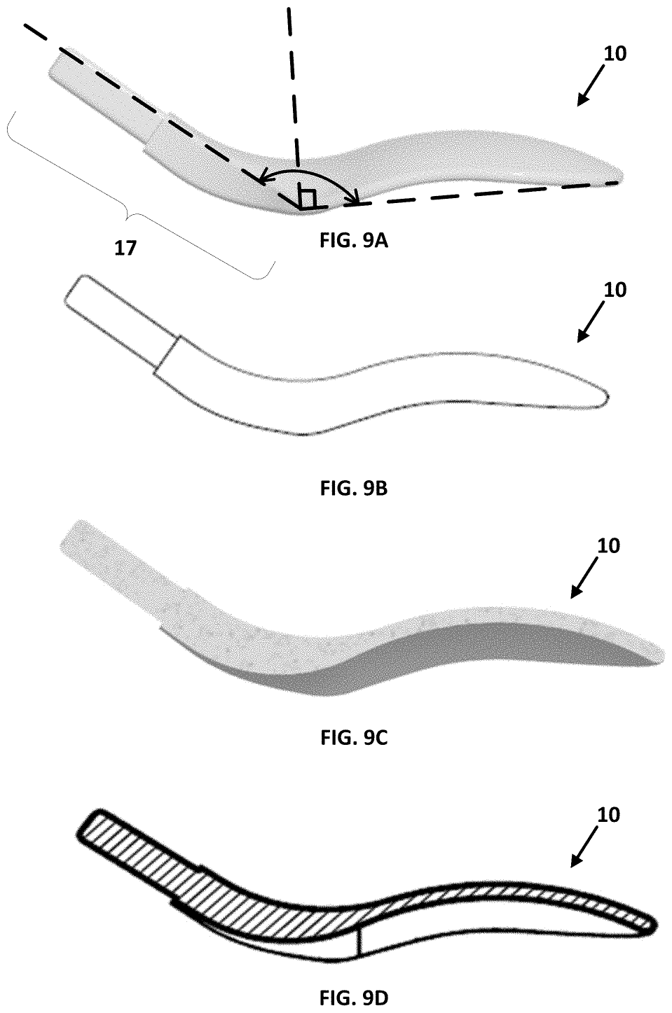

In this embodiment, the upwardly-facing surface 13 of contact portion 12 is the surface intended to contact the underside of an eyelid EL, and is convex both along the distal-proximal direction (right to left in FIG. 9A) and along the right-left direction (into the page in FIG. 9A). The arc of the convex shape is similar to, though slightly larger than, the arc of the globe G itself. In this way, upwardly-facing surface 13 of contact portion 12 can contact the underside of a region of the eyelid EL during manipulation in a way that is similar to the way that the globe G itself normally contacts the eyelid EL in that region when the eyes are closed. Similarly, the downwardly facing surface 14 of the contact portion 12 will—from time to time—contact the globe G. The downwardly facing surface 14 is, in this embodiment, concave both along the distal-proximal direction and along the left-right direction. The arc of the concave shape is similar to, though slightly larger than, the arc of the globe G itself. In this way, downwardly-facing surface 14 of contact portion 12 when occasionally contacting the globe G in a particular region can do so in a way that is similar to the way in which the eyelid EL itself normally contacts the globe G in that region when the eyes are closed. The convex and concave shaping also reduce the chance of pinching of the skin of an eyelid EL or the regions at the periphery of the eye socket during manipulation.

Handle 20 facilitates single hand operation, freeing the other hand to manipulate an RF treatment device or other device while EMD 10 is being held and manipulated using handle 20. The operability of handle 20 allows for greater facilitation, specifically in the areas surrounding the eyelid margin, which is critical in the effective treatment of Meibomian gland expression. The enhanced operability over conventional solutions decreases the overall time of Meibomian gland treatment and discomfort for the patient.

It will be understood that the size of an emmetropic adult eye is approximately 24.2 mm (transverse, horizontal)×23.7 mm (sagittal, vertical)×22.0-24.8 mm (axial, anteroposterior) with minimal difference between sex and age. In the transverse diameter, the eyeball size may vary from 21 mm to 27 mm. It will be understood that myopia and hypermetropia change the axial diameter significantly leading to variance ranging from 20 to 26 mm.

In this embodiment, the contact portion of EMD 10 has a transverse diameter of 30 mm, thereby to be larger than a human eye, thus reducing the potential for the edges to become the point of contact during normal use. A tip width of 20 mm provides a wide enough surface to facilitate eyelid expression without compromising comfort of the patient.

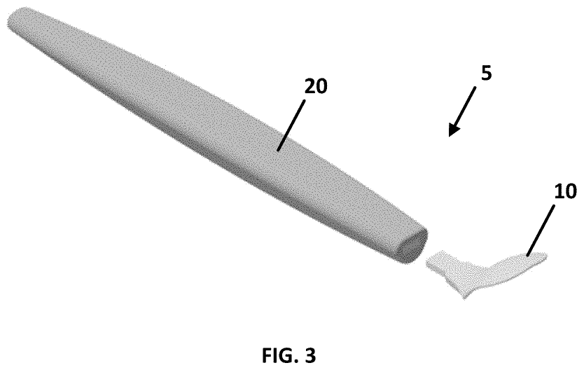

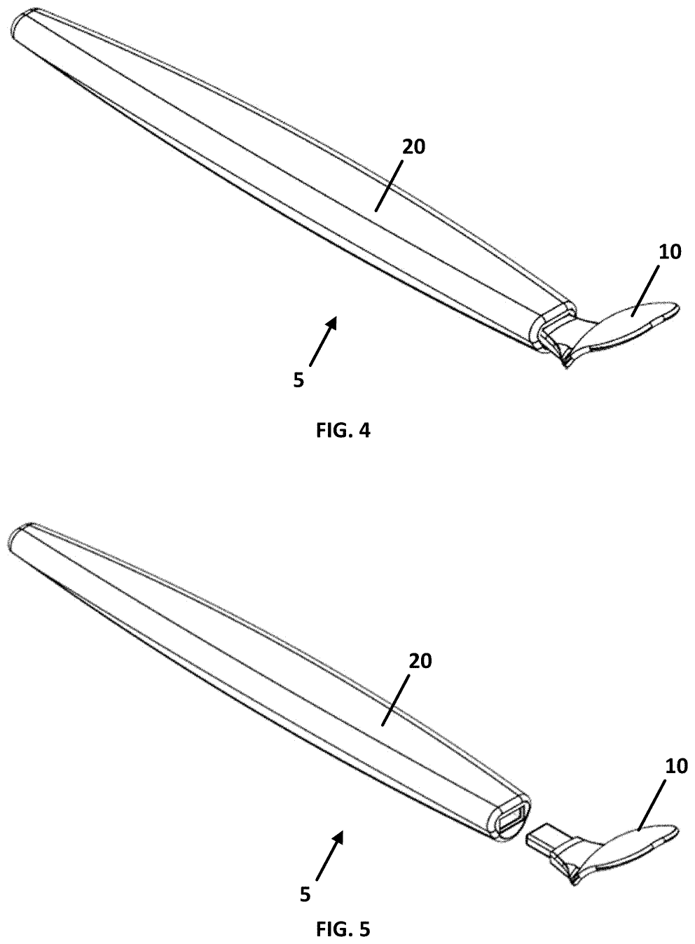

FIG. 3 is another front perspective rendered view of EMD 10 shown separated from handle 20. Stem 17, which in this embodiment is rectangular in cross-section, can be inserted into a complementary rectangular slot 21 in the end of handle 20 and held in place during use in interference fit. In this embodiment, slot 21 is tapered so that when stem 17 is received in slot 21 the friction fit is increased the farther stem 17 is pushed into slot 21. At least the interior of slot 21 is formed of a slightly harder material than that of stem 17, so that stem 17 yields slightly when forced into slot 21 until a shoulder of stem 17 (best viewed in FIGS. 5, 7A, 8B, 10B, and 11B, though shown elsewhere) meets the end of handle 20. In this embodiment, the interference fit spans axially about 4 mm, and requires at least 20 pounds of force to pull handle 20 and stem 17 apart.

In this embodiment, handle 20 is generally triangular in cross-section—with slightly rounded edges. The generally triangular shape cross-section first widens and then tapers along the length from distal end to proximal end. Handles with different cross sectional shapes such as those that are the same cross-sectional size from distal end to proximal end, may alternatively be used. Handles having longer or shorter lengths may be used. An elongate handle of the relative size shown can be useful, since a user of EMD 10 may wish to hold handle 20 near its proximal end, near its distal end, or somewhere in between, in order to manipulate in different ways for a given treatment or in different ways for different treatments. In embodiments, handle 20 may be coated with a material helpful for improving manual grip, such as medical grade silicone and/or some other material suitable in a medical context. Polyethylene may be considered.

FIG. 4 is a front perspective wireframe view of EMD 10 connected to handle 20. FIG. 5 is a front perspective wireframe view of EMD 10 separated from handle 20. These figures are similar to FIGS. 2 and 3, respectively, with the wireframe presentation providing additional visual guidance as to the surfaces and interfaces between surfaces.

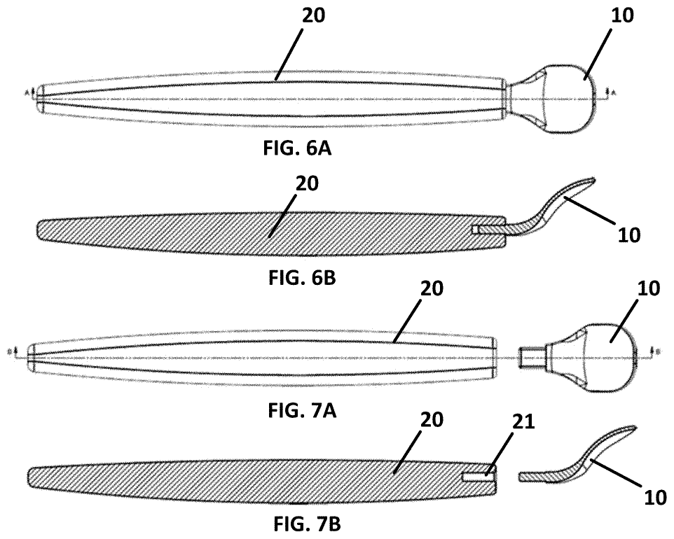

FIG. 6A is a top plan wireframe view of EMD 10 connected to handle 20. This view shows the upwardly facing surface 13 of contact portion 12 of EMD 10. FIG. 6B is a side view of EMD 10, shown sectioned along the line A-A in FIG. 6A. This view shows the convex and concave arcs of the upwardly facing surface 13 and downwardly facing surface 14 of contact portion 12, and stem 17 extending upwardly (leftward in FIG. 6B) from proximal end 15 of contact portion 12. FIG. 7A is a top plan wireframe view of EMD 10 separate from handle 20. FIG. 7B is a side view of EMD 10, shown sectioned along the line B-B in FIG. 7A.

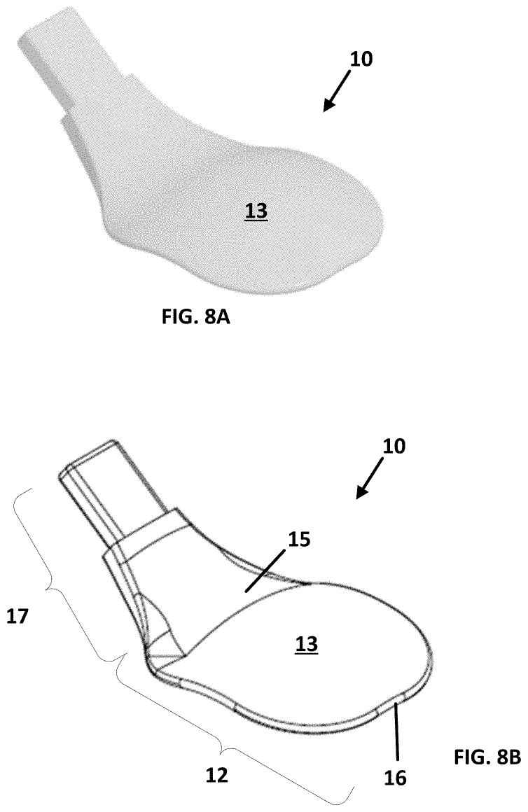

FIG. 8A is a front perspective rendered view of EMD 10 in isolation. FIG. 8B is a front perspective wireframe view of EMD 10 in isolation. FIG. 8B is similar to FIG. 8A, with the wireframe presentation providing additional guidance as to the surfaces and interfaces between surfaces. These front perspective views show the convex arc of the upwardly facing surface in both distal-proximal and left-right directions.

FIG. 9A is a side elevation rendered view of EMD 10 in isolation. FIG. 9B is a side elevation wireframe view of EMD 10 in isolation. FIG. 9C is a side section rendered view of EMD 10 in isolation. FIG. 9D is a side section wireframe view of EMD 10 in isolation. The wireframe presentations are similar to the rendered presentations, but provide additional visual guidance as to the surfaces and interfaces between the surfaces. It can be seen from the dashed angle lines in FIG. 9A that stem 17 extends at an angle greater than 90 degrees from a notional straight line connecting the distal and proximal extents of downwardly facing surface 14 of contact portion 12. In this way, stem 17 extends “out of the way” so that the user is not required to manipulate an eyelid EL in alignment with the axis of globe G. This provides an advantage in that the user is less likely to have his or her own view of eyelid EL occluded by his or her own hand holding handle 20. That is, the user can manipulate the contact portion 12 from an “off-axis” location. In this embodiment, the angle at which the stem extends is about 135 degrees. In alternative embodiments, the angle may be somewhat greater than 135 degrees, or somewhat less than 135 degrees. Furthermore, it may in fact be useful for users to work with stems that extend at 90 degrees. In alternative embodiments, a given handle may be configured to interface with stem 17 in a way that the handle extends from stem 17 at a different angle from which stem 17 extends from contact portion 12. For example, a given handle might have a bend, or be entirely linear but with an angled slot, enabling the manipulations to be done off axis despite, for example, stem 17 extending at 90 degrees. Variations are possible.

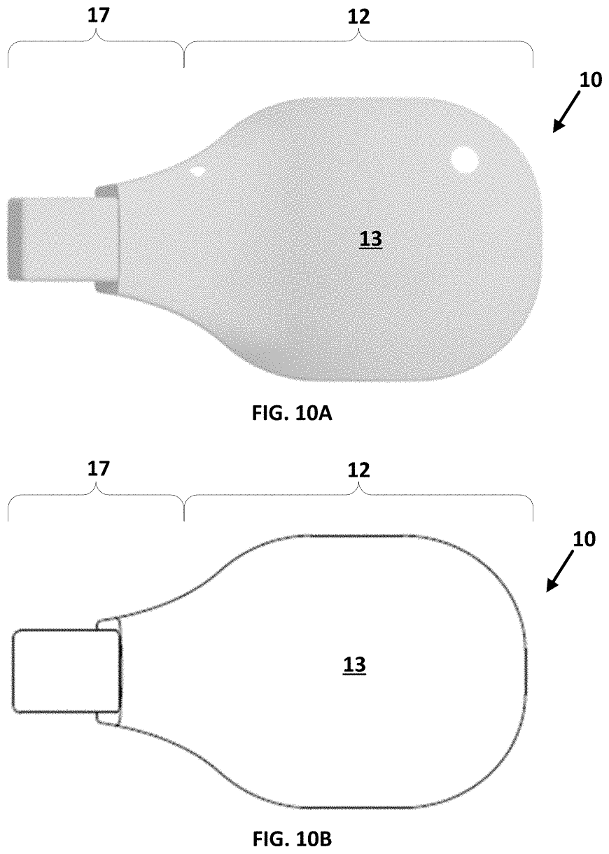

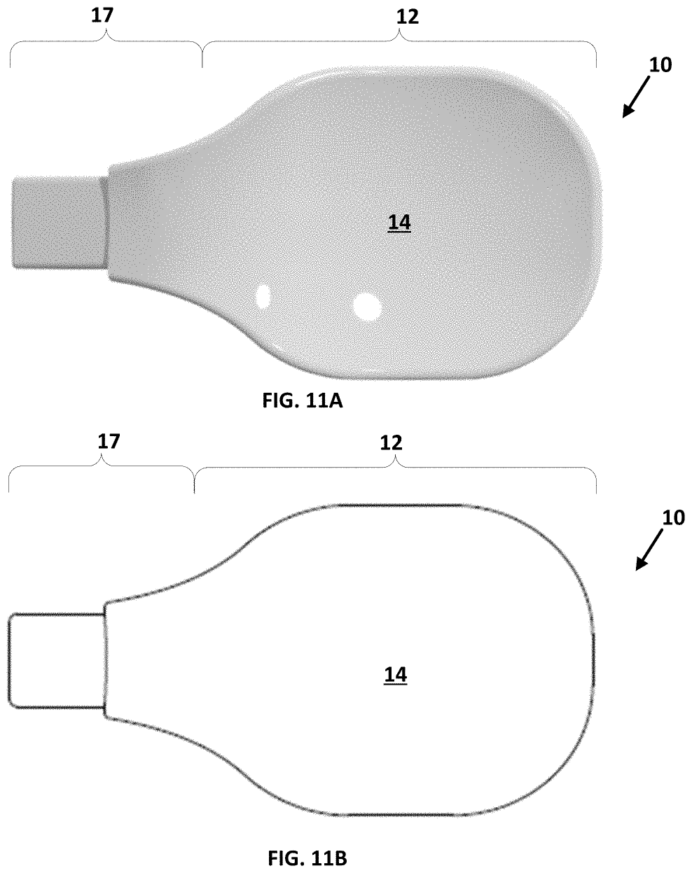

FIG. 10A is a top rendered view of EMD 10 in isolation. FIG. 10B is a top wireframe view of EMD 10 in isolation. The wireframe presentation is similar to the rendered presentation, but provides additional visual guidance as to the surfaces and interfaces between the surfaces.

FIG. 11A is a bottom rendered view of EMD 10 in isolation. FIG. 11B is a bottom wireframe view of EMD 10 in isolation. These views show the concave (into the page) downwardly facing surface of contact portion 12 of EMD 10. The wireframe presentation is similar to the rendered presentation, but provides additional visual guidance as to the surfaces and interfaces between the surfaces.

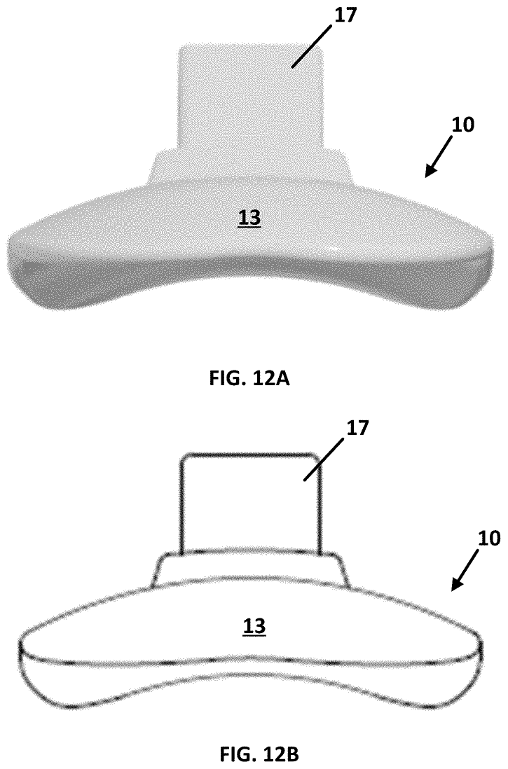

FIG. 12A is a distal end rendered view of EMD 10 in isolation. FIG. 12B is a distal end wireframe view of EMD 10 in isolation. The distal end 16 of EMD 10 is the end of its contact portion 12 that a user causes to enter first between an eyelid EL and its globe G. The wireframe presentation is similar to the rendered presentation, but provides additional visual guidance as to the surfaces and interfaces between the surfaces.

It will be appreciated from the drawings that the interfaces between the various sides and surfaces are rounded sufficiently so that EMD 10 does not present sharp corners or edges to the eyelid and eye during use.

In embodiments described above, handle 20 and EMD 10 are not integral, so that they can be connected for use and then separated, with the EMD 10 potentially being disposed of without necessarily having to dispose of handle 20. In this way, handle 20 can be re-used, and the EMDs 10 can be disposed of after a given use. This also enables one handle to be used with various different thicknesses or other configurations of EMDs.

EMDs with different thicknesses may be provided. For example, EMDs with contact portions having smaller or larger thicknesses may be chosen by a user for a particular application and/or as a preference. For example, the maximum thickness of the contact portion of a given EMD may be 1.00 mm, 1.25 mm, 1.5 mm, 1.75 mm, or larger or smaller sizes. One factor in the thickness will be the ease with which the contact portion 12 can be inserted between eyelid EL and globe G. Another will be the utility of the corneal shielding that contact portion 12 can provide. For example, if the material of a given contact portion 12 is dense, or particularly suited to shielding from RF radiation, it may be thinner than another contact portion of another EMD made of a material that is not very dense or otherwise suited to shielding at small thicknesses. In addition, a contact portion of an EMD may be coated with a material that is both excellent for RF shielding as well as suitable for use in a medical context, thereby to supplement the shielding provided by the underlying contact portion.

In use, a user such as a medical practitioner inserts the stem of an EMD 10 into a handle 20 in interference fit, checks the fit, and then with the aid of handle 20 gently slides the distal end 16 of EMD 10 between an eyelid EL and the globe G beneath it. The eyelid EL and globe G may have previously been treated with an appropriate lubricant. With EMD 10 having been inserted between the eyelid EL and globe G, the user can then slightly lift eyelid EL by slightly pulling EMD 10 away from the globe G using handle 20. Then, if the user wishes, he or she can slide EMD 10 back and forth underneath the eyelid EL to target manipulation to particular regions of the eyelid EL, and can draw the eyelid EL away from the globe G in any of these regions thereby to focus sufficient pressure on the underside of the eyelid EL in the selected region. The user can then apply RF radiation to the other side of the eyelid region thereby to treat the Meibomian glands. The user can also gently move an EMD 10 from side to side and front to back, as desired, in order to impart pressure on the region in different ways and in different amounts, thereby to effect gland expression. The user may wish to twist handle 20 slightly to, via EMD 10, apply edge pressure to the underside of the eyelid EL in the selected region, if desired, while accordingly adjusting the position of the source of RF radiation thereby to ensure that the globe G continues to be sufficiently shielded.

FIGS. 13A, 13B and 13C are side sectional schematic views of the eyelid EL and globe G of a leftward-facing individual, with the contact portion of an EMD 10 being insinuated between the eyelid EL and the globe G and then being manipulated to lift a region of the eyelid EL upwardly and away from the globe G for a treatment of that region.

Although embodiments have been described with reference to the drawings, those of skill in the art will appreciate that variations and modifications may be made without departing from the spirit, scope and purpose of the invention as defined by the appended claims.

For example, while in embodiments described the surfaces of the contact portion are arced in both the distal-proximal and left-right directions, alternatives are possible in which the arcing is in only one of these directions. Furthermore, provided edges and corners are rounded, an alternative contact portion might have generally planar upwardly facing and/or downwardly facing surfaces.

Furthermore, alternatives in which the stem is elongated and thus itself serves as an integral handle are possible. The stem may, along its length, change in cross-sectional shape thereby to serve more suitably as a stem at the proximal end of the rigid contact portion, and more suitably as a handle as it extends away from the rigid contact portion.

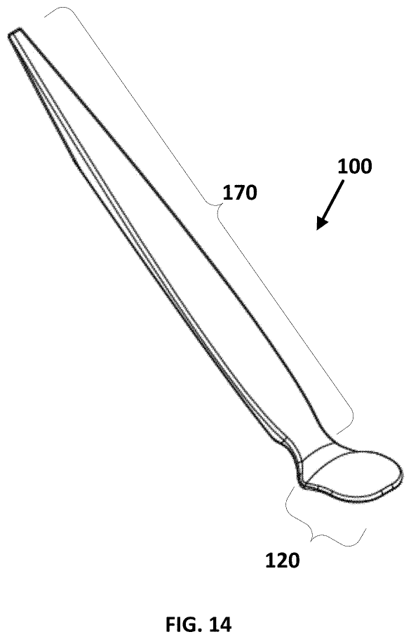

For example, FIG. 14 is a front perspective rendered view of an EMD 100, according to another embodiment. The configuration of EMD 100 is similar to a combination of EMD 10 and handle 20 described above. However, EMD 100 includes a contact portion 120 that is integral with an elongated stem/handle 170 such that EMD 100 is a single-piece unit. Configuration, dimensions, relative dimensions, curvatures, and the like, are just as described above in connection with the “two-piece” eyelid manipulation system 5 and its variations, except that in the embodiment of FIG. 14, contact portion 120 and stem/handle 170 are unitary i.e. are created/molded as a single-piece unit. After use of EMD 100, the entire single-piece EMD 100 may be discarded or cleaned or otherwise handled as one unit. It will be appreciated that a single-piece unit such as EMD 100 may be easier for a user to make use of due to it not having to be assembled prior to use by the user as might be the case with a two-piece system such as is described above. This would reduce the risks that might be associated with improper or insufficient assembly, which could occur if a user was in a hurry or not being careful, and which could lead to improper use, or disassembly during use for manipulation of an eyelid.

What is claimed is:

1. An eyelid manipulation device comprising:
a rigid contact portion dimensioned to be insinuated between an eyelid and a globe, the contact portion having an upwardly-facing surface opposite a downwardly-facing surface, each of the surfaces extending between a distal side and a proximal side of the contact portion; and
a rigid stem integral with and extending upwardly from the proximal side of the contact portion.

2. The eyelid manipulation device of claim 1, wherein at least the exterior of the eyelid manipulation device is made of at least one non-conductive material.

3. The eyelid manipulation device of claim 2, wherein the at least one non-conductive material comprises a medical grade non-conductive material.

4. The eyelid manipulation device of claim 3, wherein the medical grade non-conductive material is selected from the group consisting of: silicone and polyethylene.

5. The eyelid manipulation device of claim 1, wherein the stem is connectable to a handle.

6. An eyelid manipulation system comprising:
the eyelid manipulation device of claim 5; and
an elongate handle removably connectable to the rigid stem.

7. The eyelid manipulation system of claim 6, wherein at least the exterior of the handle is formed of at least one non-conducting material.

8. Use of the eyelid manipulation system of claim 6 for treatment of Meibomian glands.

9. Use of the eyelid manipulation system of claim 6 for treatment of dry eye symptoms.

10. The eyelid manipulation device of claim 1, wherein the stem is elongated, thereby serving as a handle.

11. Use of the eyelid manipulation device of claim 10 for treatment of Meibomian glands.

12. Use of the eyelid manipulation system of claim 10 for treatment of dry eye symptoms.
